(19) United States
(12) Patent Application Publication
(10) Pub. No.: US 2023/0282369 A1
(43) Pub. Date: Sep. 7, 2023

(54) CARE MANAGEMENT SYSTEM

(71) Applicant: Home Services Collaboration, LLC, Cincinnati, OH (US)

(72) Inventors: Stephen Rosedale, Blue Ash, OH (US); Ronald Wilheim, Blue Ash, OH (US)

(73) Assignee: Home Services Collaboration, LLC, Cincinnati, OH (US)

(21) Appl. No.: 17/685,224

(22) Filed: Mar. 2, 2022

(51) Int. Cl. G16H 80/00; G16H 40/67; G16H 10/60

(52) U.S. Cl. CPC G16H 80/00; G16H 10/60; G16H 40/67

(57) ABSTRACT

In certain aspects, a computer-implemented method is includes receiving a request from a user device comprising a user profile comprising medical and social information of a user associated with the user device. The method includes adding, responsive to receiving the request, the user device to a queue. The method includes selecting a personal care partner device to connect with the user device based on care parameters. The method includes displaying, on the personal care partner device, selected information of the medical and social information responsive to the personal care partner device being selected. The method includes connecting, responsive to the personal care partner device accepting the request, the user device to the personal care partner device. The method includes generating a log of monitored information received from the personal care partner device and the user device, and includes updating the user profile with the log of monitored information.

### TECHNICAL FIELD

The present disclosure generally relates to management systems, and more specifically relates to care management systems.

### BACKGROUND

Individual health management is a manageable task, but often becomes more difficult as the individual ages. For example, the individual’s health or medical conditions may increase from year to year requiring coordination between more providers and specialists. While some traditional technologies attempt to streamline appointment scheduling with multiple providers and monitor health issues of the individual, these technologies typically overlook the impact of the individual’s social life, including everyday activities, on health related subjects. Furthermore, such technologies may present usage difficulties for the aging individual who may lack the technological literacy required for navigating and operating the devices supporting these technologies.

The description provided in the background section should not be assumed to be prior art merely because it is mentioned in or associated with the background section. The background section may include information that describes one or more aspects of the subject technology.

### SUMMARY

The present disclosure provides systems and methods for providing personalized patient support and guidance based on secured medical and social information.

According to certain aspects of the present disclosure, a computer-implemented method is provided. The method includes receiving, at a care management server, a call request from a user device comprising a user profile, wherein the user profile comprises medical and social information of a user associated with the user device. The method includes adding, responsive to receiving the call request, the user device to a queue. The method includes selecting a personal care partner device of a plurality of care partner devices to connect with the user device added in the queue based on care parameters. The method includes displaying, on the personal care partner device, selected information of the medical and social information of the user profile responsive to the personal care partner device being selected to connect with the user device. The method includes connecting, responsive to the personal care partner device accepting the call request, the user device to the personal care partner device on a call. The method includes generating a log of monitored information received from the personal care partner device and the user device during the call. The method includes updating the user profile with the log of monitored information.

According to other aspects of the present disclosure, a system is provided. The system includes a memory comprising instructions and a processor configured to execute the instructions which, when executed, cause the processor to receive, at a care management server, a call request from a user device comprising a user profile, wherein the user profile comprises medical and social information of a user associated with the user device. The processor is configured to execute the instructions which, when executed, cause the processor to add, responsive to receiving the call request, the user device to a queue. The processor is configured to execute the instructions which, when executed, cause the processor to select a personal care partner device of a plurality of personal care partner devices to connect with the user device added in the queue based on care parameters. The processor is configured to execute the instructions which, when executed, cause the processor to display, on the personal care partner device, selected information of the medical and social information of the user profile responsive to the personal care partner device selected to connect with the user device. The processor is configured to execute the instructions which, when executed, cause the processor to connect, responsive to the personal care partner device accepting the call request, the user device to the personal care partner device on a call. The processor is configured to execute the instructions which, when executed, cause the processor to generate a log of monitored information received from the personal care partner device and the user device during the call. The processor is configured to execute the instructions which, when executed, cause the processor to update the user profile with the log of monitored information.

According to other aspects of the present disclosure, a non-transitory machine-readable storage medium comprising machine-readable instructions for causing a processor to execute a method is provided. The method includes receiving, at a care management server, a call request from a user device comprising a user profile, wherein the user profile comprises medical and social information of a user associated with the user device. The method includes adding, responsive to receiving the call request, the user device to a queue. The method includes selecting a personal care partner device of a plurality of care partner devices to connect with the user device added in the queue based on care parameters. The method includes displaying, on the personal care partner device, selected information of the medical and social information of the user profile responsive to the personal care partner device being selected to connect with the user device. The method includes connecting, responsive to the personal care partner device accepting the call request, the user device to the personal care partner device on a call. The method includes generating a log of monitored information received from the personal care partner device and the user device during the call. The method includes updating the user profile with the log of monitored information. The method includes accessing prior to receiving the call request, from an electronic medical record server, an electronic medical record associated with the user of the user device. The method includes integrating information of the electronic medical record associated with the user into the user profile.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. It should be noted that although various aspects may be described herein with reference to particular settings, these are examples only and are not to be considered limiting. The teachings of the present disclosure may be applied to other mobile device environments, including but not limited to healthcare environments, telehealth environments, telemedicine environments, independent and assisted living environments, home environment, and other organizational environments well-known in the industry. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example architecture for providing personalized patient support and guidance based on secured medical and social information.

FIG. 2 is a block diagram illustrating an example care management server, emergency medical service server, electronic medical record server, at least one user device, at least one personal care partner device, at least one provider device, and at least one companion device from the architecture of FIG. 1 according to certain aspects of the disclosure.

FIG. 3 illustrates an example process for using the care management server, the electronic medical record server, the at least one user device, and the at least one care partner device of FIG. 2.

FIGS. 4A-4F are example illustrations associated with the example process of FIG. 3.

FIG. 5 is block diagram illustrating an example computer system with which the example care management server, emergency medical service server, electronic medical record server, at least one user device, at least one personal care partner device, at least one provider device, and at least one companion device of FIG. 2 can be implemented.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

The disclosed systems and methods provide solutions to traditional remote monitoring systems for healthcare, which merely monitor user’s data and relay health information, as well as to traditional telehealth systems. For example, the disclosed systems and methods can receive user information, including medical and social information, to create a user profile and associate the user profile to a unique user device, which enables the user device to securely communicate with a personal care partner on a personal care partner device via a care management server. The personal care partner can access the user profile moments before communicating with the user on the user device so the personal care partner can be acquainted with the user profile, which is periodically updated with analytics of the user information and user interaction on the user device, in order to provide personal support and guidance specific to the user. For example, the personal care partner on the personal care partner device can determine the needs of the user and can connect the user device to a provider device for communication with a provider.

The disclosed systems and methods provide an improvement to computer functionality by allowing computer performance of a function not previously performed by a computer arising in the realm of care management. The disclosed systems and methods improve care management technology in a variety of manners including, but not limited to, compiling and utilizing secured medical and social information to provide personalized patient support and guidance based on such information to understand patient needs such that a patient, via a user device, is routed to an appropriate personal care partner, via a personal care partner device, based on care parameters.

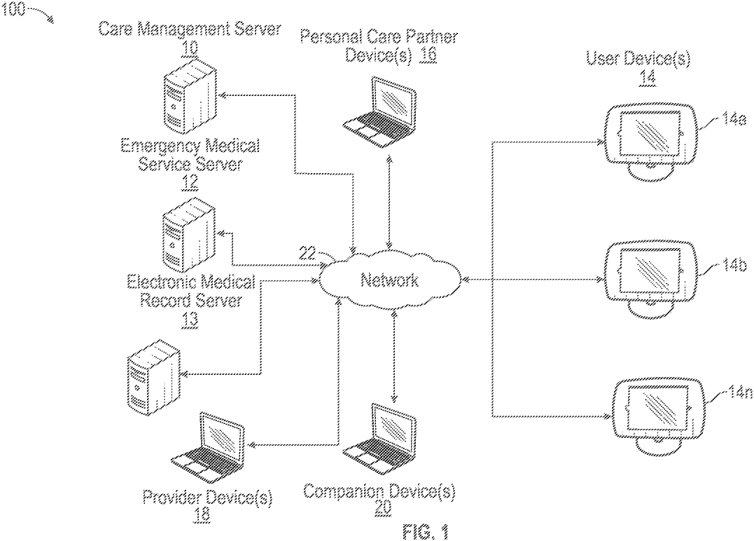

FIG. 1 illustrates an example architecture 100 for providing personalized patient support and guidance based on secured medical and social information. For example, the architecture 100 includes a care management server 10, an emergency medical service server 12, an electronic medical record server 13, at least one user device 14, such as user devices 14a, 14b... 14n, at least one personal care partner device 16, at least one provider device 18, and at least one companion device 20 all connected over a network 22.

The mobile device management server 10 can be any device having an appropriate processor, memory, and communications capability for communicating with the emergency medical service server 12, the electronic medical record server 13, the at least one user device 14, such as the user devices 14a, 14b... 14n, the at least one personal care partner device 16, the at least one provider device 18, and the at least one companion device 20. For purposes of load balancing, the care management server 10 may include multiple servers. The emergency medical service server 12 can be any device having an appropriate processor, memory, and communications capability for communicating with the care management server 10 and the at least one user device 14. The electronic medical record server 13 can be any device having an appropriate processor, memory, and communications capability for communicating with the care management server 10, the at least one user device 14, such as the user devices 14a, 14b... 14n, the at least one personal care partner device 16, the at least one provider device 18, and the at least one companion device 20. In certain aspects, the care management server 10 and the emergency medical service server 12 can be a cloud computing server of an infrastructure-as-a-service (IaaS) and be able to support a platform-as-a-service (PaaS) and software-as-a-service (SaaS) services.

The at least one user device 14, such as the user devices 14a, 14b...14n, to which the care management server 10, the emergency medical service server 12, the electronic medical record server 13, the at least one personal care partner device 16, the at least one provider device 18, and the at least one companion device 20 communicates with over the network 22, can be, for example, a tablet computer, a mobile phone, a mobile computer, a laptop computer, a portable media player, an electronic book (eBook) reader, a wearable device, or any other device having appropriate processor, memory, and communications capabilities.

The at least one personal care partner device 16, to which the emergency medical service server 12, the electronic medical record server 13, the at least one user device 14, such as the user devices 14a, 14b...14n, the at least one personal care partner device 16, the at least one provider device 18, and the at least one companion device 20 communicates with over the network 22, can be, for example, a tablet computer, a mobile phone, a mobile computer, a laptop computer, a portable media player, an electronic book (eBook) reader, or any other device having appropriate processor, memory, and communications capabilities.

The at least one provider device 18, to which the care management server 10, the electronic medical record server 13, the at least one user device 14, such as the user devices 14a, 14b... 14n, the at least one personal care partner device 16, and the at least one companion device 20 communicates with over the network 22, can be, for example, a tablet computer, a mobile phone, a mobile computer, a laptop computer, a portable media player, an electronic book (eBook) reader, or any other device having appropriate processor, memory, and communications capabilities.

The at least one companion device 20, to which the care management server 10, the at least one user device 14, such as the user devices 14a, 14b...14n, the at least one personal care partner device 16, and the at least one provider device 18 communicates with over the network 22, can be, for example, a tablet computer, a mobile phone, a mobile computer, a laptop computer, a portable media player, an electronic book (eBook) reader, or any other device having appropriate processor, memory, and communications capabilities.

It should be noted that the present disclosure is not limited to any particular configuration or number of user devices, such as the user devices 14a, 14b... 14n. In certain aspects, a different number of managed devices may be present.

The network 22 can include, for example, any one or more of a personal area network (PAN), a local area network (LAN), a campus area network (CAN), a metropolitan area network (MAN), a wide area network (WAN), a broadband network (BBN), the Internet, and the like. Further, the network 22 can include, but is not limited to, any one or more of the following network topologies, including a bus network, a star network, a ring network, a mesh network, a star-bus network, tree or hierarchical network, and the like.

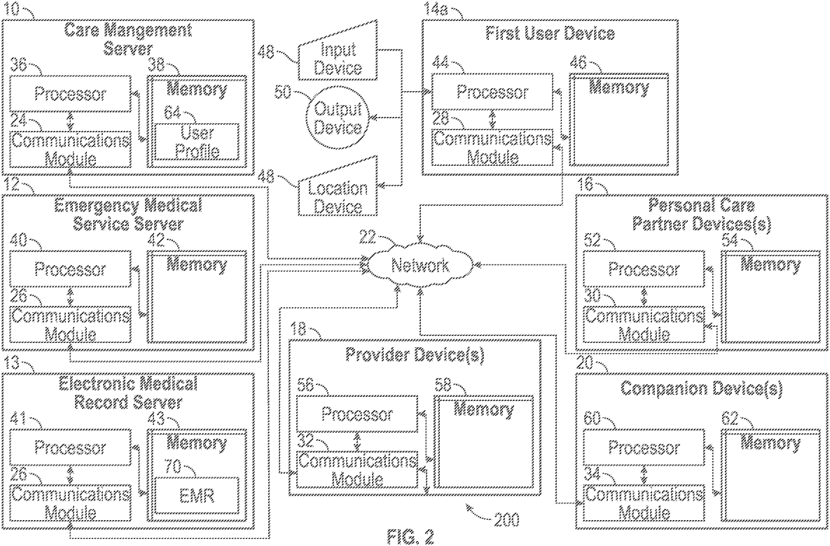

FIG. 2 is a block diagram illustrating examples of the care management server 10, the emergency medical service server 12, the electronic medical record server 13, the at least one user device 14, such as the user devices, the at least one personal care partner device 16, the at least one provider device 18, and the at least one companion device 20, in the architecture of FIG. 1 according to certain aspects of the disclosure. It should be understood that while the care management server 10 monitors and communicates with a plurality of user devices, such as the at least one user device 14, the first user device 14a will be discussed in particular to simplify description of the present disclosure.

The care management server 10, the emergency medical service server 12, the at least one user device 14, such as the first user device 14a, the at least one personal care partner device 16, the at least one provider device 18, and the at least one companion device 20 are connected over the network 22 via respective communications modules 24, 26, 27, 28, 30, 32, 34. The communications modules 24, 26, 27, 28, 30, 32, 34 are configured to interface with the network 22 to send and receive information, such as data, requests, responses, and commands to other devices on the network 22. The communications modules 24, 26, 27, 28, 30, 32, 34 can be, for example, modems or Ethernet cards.

The care management server 10 includes a processor 36, the communications module 24, and a memory 38. The processor 36 of the mobile device management server 10 is configured to execute instructions, such as instructions physically coded into the processor 36, instructions received from software in the memory 38, or a combination of both. The mobile device management server 10 may correspond to hardware and/or software that implement mobile device management functions. Although the care management server 10 is illustrated as a single server for ease of discussion, it should be understood that the care management server 10 can be multiple servers in communication with one another.

The emergency medical service server 12 includes a processor 40, the communications module 26, and a memory 42. The processor 40 of the emergency medical service server 12 is configured to execute instructions, such as instructions physically coded into the processor 40, instructions received from software in the memory 42, or a combination of both.

The electronic medical record server 13 includes a processor 41, the communications module 27, and a memory 43. The processor 41 of the electronic medical record server 13 is configured to execute instructions, such as instructions physically coded into the processor 41, instructions received from software in the memory 43, or a combination of both.

The first user device 14a includes a processor 44, the communications module 28, and a memory 46. The processor 44 of the first user device 14a is configured to execute instructions, such as instructions physically coded into the processor 44, instructions received from software in memory 46, or a combination of both. The first user device 14a also includes an input device 48, such as a touch screen, and an output device 50, such as a display. The first user device 14a includes a location device 51 configured to transmit the location of the first user device 14a to the care management server 10. The location device 51 can identify the location of the first user device 14a by utilizing any positioning system well-known in the industry such as, but not limited to, global positioning system (GPS), cellular based positioning systems, beacon based technology systems, and other well-known positioning systems.

The at least one personal care partner device 16 includes a processor 52, the communications module 30, and a memory 54. The processor 52 of the at least one personal care partner device 16 is configured to execute instructions, such as instructions physically coded into the processor 52, instructions received from software in memory 54, or a combination of both.

The at least one provider device 18 includes a processor 56, the communications module 32, and a memory 58. The processor 56 of the at least one provider device 18 is configured to execute instructions, such as instructions physically coded into the processor 56, instructions received from software in memory 58, or a combination of both.

The at least one companion device 20 includes a processor 60, the communications module 34, and a memory 62. The processor 60 of the at least one companion device 20 is configured to execute instructions, such as instructions physically coded into the processor 60, instructions received from software in memory 62, or a combination of both.

The care management server 10 is configured to monitor the first user device 14a as well as connect the first user device 14a to other devices on the network 22 such as the at least one personal care partner device 16, the at least one provider device 18, the at least one companion device 20, and the emergency medical service server 12. For example, the care management server 10 can monitor and record user interactions and usage of the first user device 14a such as, but not limited to, uptime activity, downtime inactivity, service type usage, response times, call types, calendar appointments, user feedback, and other types of interactions and usage. The care management server 10 can also monitor software versions on the first user device 14a and transmit upgrades to the first user device 14a to update the software when appropriate.

The care management server 10 is configured to generate a user profile 64 based on intake information such as medical information 66 and social information 68. The medical information 66 can include demographic information of the user such as, but not limited to, name, date of birth, gender, address, telephone number, emergency call back phone number, email address, insurance information, race/ethnicity, emergency contact. The medical information 66 can include provider information of the user such as, but not limited to, primary care physician name, primary care physician address, primary care physician phone number, specialist name, specialist address, specialist phone number, pharmacy name, pharmacy address, pharmacy phone number, preferred lab name, preferred lab address, preferred lab phone number, and other provider information. The medical information 66 can include medication information such as, but not limited to, drug name, dosage, frequency, diagnosis prescribed to treat, affordability issues, self-administration of drug capability. The medical information 66 can include allergy information such as, but not limited to, allergy type, drug name user is allergic to, and reaction to drug. The medical information 66 can include medical history information such as, but not limited to, cardiovascular history, pulmonary history, gastrointestinal history, muscular history, skeletal history, skin history, cancer history, behavioral history, endocrine history, cystic breast disease history for women, menorrhagia history for women, amenorrhea history for women, polycytic overian disease history for women, sense history (e.g., hearing, vision, taste, touch, smell), chronic pain history. The medical information 66 can include special treatment information such as, but not limited to, treatments including chemotherapy, radiation therapy, home oxygen, tracheostomy, suction, ventilation, CPAP/BiPAP, IV fluids, enteral feedings, hospice, dialysis, and respite. The medical information 66 can include durable medical equipment information such as, but not limited to, use of a walker, a wheelchair, a lift, a nebulizer, a TENS unit, a hospital bed, and a wound vacuum. The medical information 66 can include hospitalization information such as, but not limited to, hospitalization history, past emergency room admissions diagnosis, date of emergency room admission, and facility name of the emergency room admission. The medical information 66 can include dietary information such as, but not limited to, dietary restrictions related to a diabetic diet, a dash diet, a renal diet, a lipid diet, a high fiber diet, and other diets. The medical information 66 can include surgical history information such as surgical history including, but not limited to, brain and nerves; eye; ears, nose, and throat; neck; chest, hear, and lungs; esophagus and stomach; gallbladder, liver, spleen, and pancreas; bowel and rectal; kidney, ureter, and bladder; breast, ovaries, and uterus for women; prostate and testicles for men; back, bone, and joints; fractured bone; and skin. The medical information 66 can include preventative care screening information including, but not limited to, healthcare effectiveness data and information set (HEDIS), health outcomes survey (HOS), and consumer assessment of healthcare and provider survey (CAHPS).

The social information 68 can include personal history such as, but not limited to, occupation history, relationship status, dependent status including number of children and grandchildren, and education status. The social information 68 can include housing information such as, but not limited to, ownership/rental information and home type. The social information 68 can include safety information such as, but not limited to, whether the user feels safe in current living environment, whether the user has ever been abused or neglected in current living environment, and whether the use has ever been sexually assaulted in current living environment. The social information 68 can include support information such as, but not limited to, who the user relies on for support including family, friends, spouse, caregiver, and other support. The social information 68 can include community information such as, but not limited to, whether the user engages with visitors, participates in volunteer work, participates in social opportunities, and other community activities. The social information 68 can include spiritual information such as, but not limited to, whether the user practices or believes in spirituality. The social information 68 can include cognitive/learning information such as, but not limited to, whether the user participates in activities including, reading, writing, hobbies, games, discovery, and observational cognitive learning. The social information 68 can include financial information such as, but not limited to, whether the user is unable to afford necessities such as rent, medications, transportation, food, and other necessities. The social information 68 can include activities of daily living (ADL) that the user participates in such as, but not limited to, toileting, communication, dressing, bathing, transfer, cooking, house cleaning, shopping, transportation, managing money, managing medication, and ability to use phone or computer.

The user profile 64 is a personalized medical and social record of the user that can be stored in the memory 38 of the care management server 10, in another storage device associated with the care management server 10, or a combination of both. The care management server 10 associates the user profile 64 specifically to the first user device 14a to enable secure communication with the at least one personal care partner device 16 via the care management server 10.

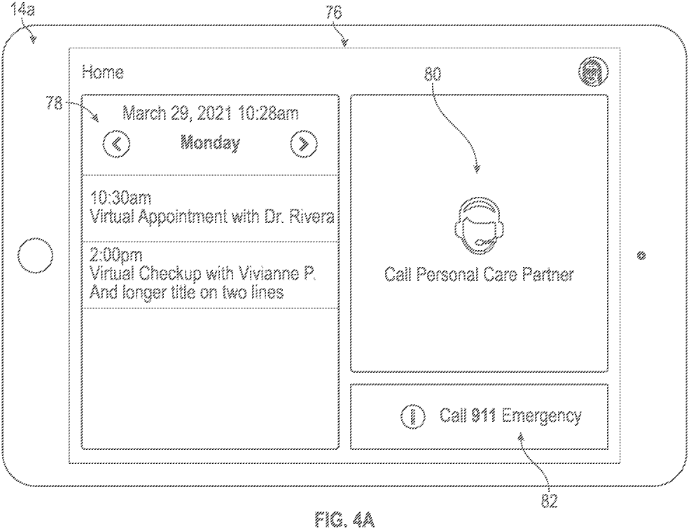

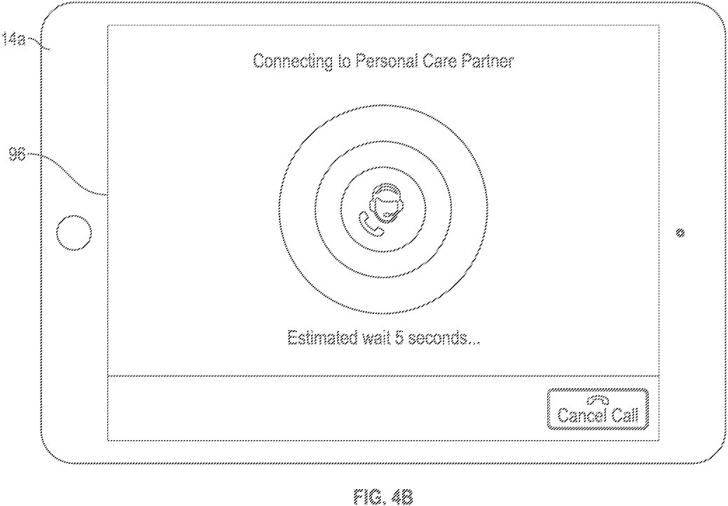

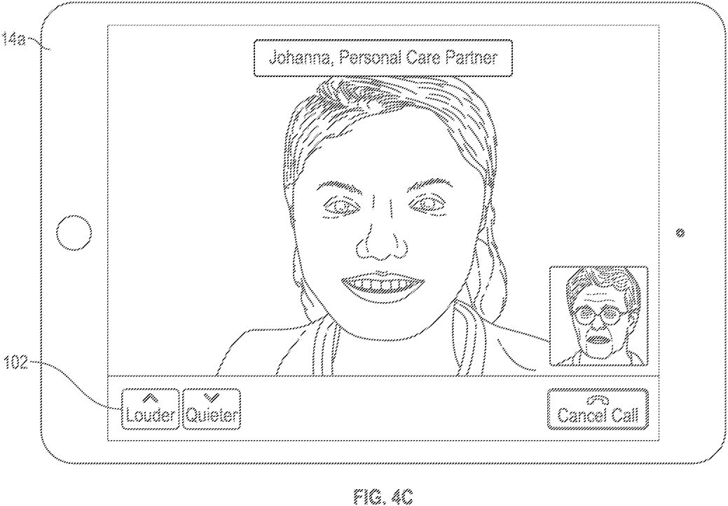

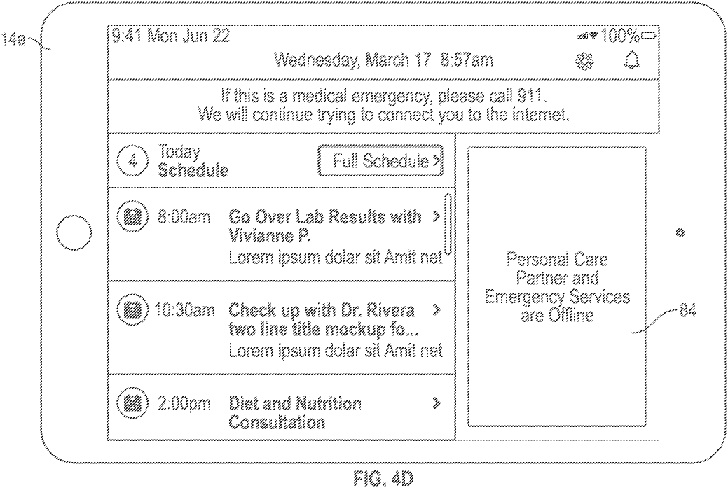

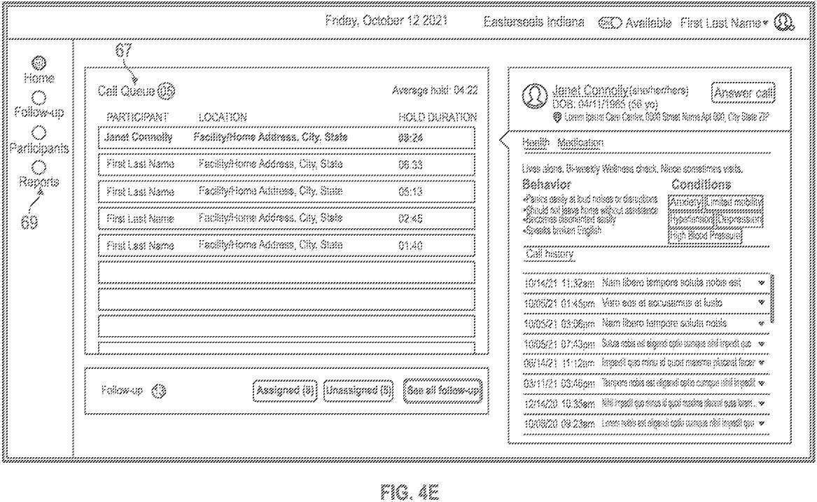

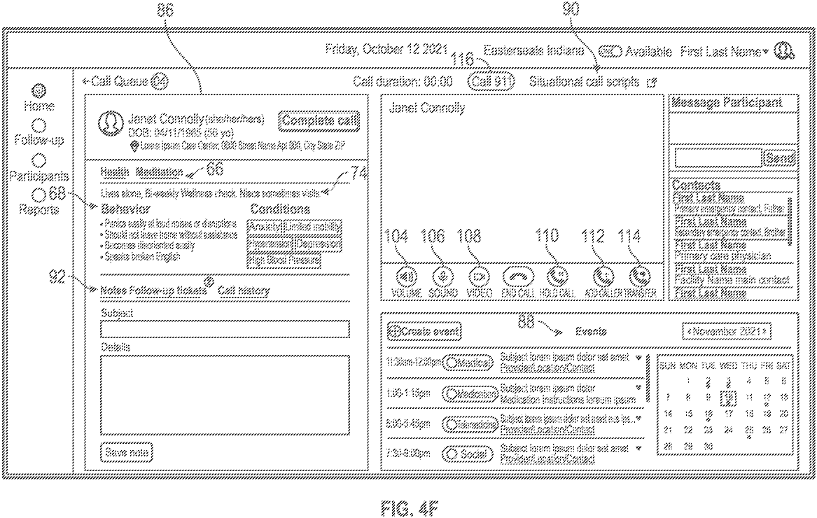

The care management server 10 is configured to receive a call request from the first user device 14a and places the first user device 14a in a queue 67 (see FIG. 4E), which may include other devices of the at least one user device 14. The care management server 10 is configured to manage the queue 67 and connect the first user device 14a to one of the at least one personal care partner device 16. In certain aspects, the care management server 10 is configured to select a personal care partner device of the at least one personal care partner device 16, based on the user profile 64 and care parameters associated with the user profile 64 of the first user device 14a in the queue 67, for connecting to the first user device 14a. The care parameters can include, but is not limited to, skills of the personal care partner associated with the least one personal care partner device 16 with respect to the user profile 64 of the user of the first user device 14a, needs of the user associated with the first user device 14a identified in the user profile 64, urgency of the call request, and other parameters that facilitate routing of the first user device 14a to an appropriate personal care partner via the at least one personal care partner device 16. The care management device 10 is configured to initiate video conferencing with the first user device 14a, the at least one personal care partner device 16, and another device such as the at least one provider device 18, the at least one companion device 20, and the emergency medical service server 12. The care management device 10 is configured to monitor and manage a calendar associated with the first user device 14a including scheduling appointments, scheduling activities, transmitting calendar reminders to the first user device 14a, transmitting assignments to the at least one personal care partner device 16 to communicate with the first user device 14a to follow up on scheduled appointments, and other such functions. The care management device 10 is configured to transmit notifications and emergency alarms to the first user device 14a.

During communication between the first user device 14a and the at least one personal care partner device 16, the care management server 10 is configured to monitor the communication and generate a log 69 of the communication, including audio files, video files, and text files of input by the personal care partner via the at least one personal care partner device 16 (e.g., notes of the call, reason for the call, follow up items, appointments, services to be used, and other call-related information), for recording and associating with the user profile 64. The care management server 10 is configured to communicate with the electronic medical service server 13 and access an electronic medical record (EMR) 70 associated with the user of the first user device 14a from the electronic medical record server 13, and is configured to integrate information of the EMR 70 into the user profile 64 associated with the user of the first user device 14a.

After a call between the first user device 14a and the at least one personal care partner device 16, the care management server 10 is configured to receive survey information 72 from the first user device 14a. The survey information 72 can include feedback information from the user of the first user device 14a about user experience, quality of call, helpfulness of call, happiness of call, and other feedback about interaction. The care management server 10 is configured to perform analytics on the survey information 72, information associated with the user profile 64, such as the log 69, and other information recorded by the care management server 10 and associated with the first user device 14a such as, for example, user interactions and usage of the first user device, uptime activity, downtime inactivity, service type usage, response times, call types, calendar appointments, emergency services used, time of call, length of call, personal care partner survey, name of personal care partner who answered call, personal care partner availability, online activity of the first user device 14a, queue wait time of the first user device 14a, servicing of follow up items, and notifications transmitted to the first user device 14a. The care management server 10 is configured to update the user profile 64 associated with the first user device 14a based on the results of the analytics to enable a personal care partner to provide personalized support and guidance to the user of the first user device 14a on future calls.

Moreover, the care management server 10 is configured to determine a persona 74 of the user of the first user device 14a based on the intake information, including the medical information 66 and the social information 68, and is configured to associate the persona 74 with the user profile 64 of the first user device 14a. The persona 74 can include various persona categories such as, but not limited to, high risk, self-sufficient, absent minded, fraternal, and social, to name a few. A high risk category of the persona 74 can include characteristics of the user such as, but not limited to, fragile health, struggling with a permanent disability or life-altering diagnosis, low proficiency of technology, stays inside, talks to family over phone, wakes up early, goes to bed early, has a close to small circle of constant caregivers that may include an aide, dependent and needs help with simple daily tasks. A self-sufficient category of the persona 74 can include characteristics of the user such as, but not limited to, strong health, struggling with hearing loss and vision loss, low proficiency of technology, uses landline phone, uses phone amplifier, goes to activities such as fitness class, gets yearly health checkups, visits friends, own caregiver, family is distant, independent and looks after self with no help. An absent minded category of the persona 74 can include characteristics of the user such as, but not limited to, suffering from memory loss, confused, and family is distant. A fraternal category of the persona 74 can include characteristics of the user such as, but not limited to, average health with some health issues, caregiver and family plays significant role in day-to-day life and care decisions, proficient with technology, uses mobile phone including applications, uses fitness tracker, goes out with children and grandchildren, gets monthly health checkups, family is close and accommodating, and dependent and enjoys closeness to caregivers - but wants to help out. A social category of the persona 74 can include characteristics of the user such as, but not limited to, below average health, struggles with issues that require medication and attention, above proficient with technology, uses mobile phone for ordering groceries, uses tablet to play games and reading, goes to see family and friends, emergency doctor visits, enjoys being part of the community, caregivers are siblings and family that are not their children, and independent - values own space.

In certain aspects, the care management server 10 is configured to transmit notifications and reminders, in advance of scheduled appointments, to the at least one user device 14. In certain aspects, the care management server 10 is configured to, responsive to a request from the at least one user device 14, connect the at least one user device 14 directly to one of the at least one provider device 16 for a scheduled appointment or on demand.

The first user device 14a of the at least one user device 14 is configured to display, via a graphical user interface (GUI) 76 (see FIG. 4A), a calendar 78, a call personal care partner button 80, and a call emergency button 82. The first user device 14a is configured to communicate with the care management server 10 in response to the user engaging with the call personal care partner button 80. The first user device 14a is configured to notify the user of notifications and reminders related to upcoming events scheduled on the calendar 78. The first user device 14a is configured to connect to the network 22 via various technologies including, but not limited to, Wi-Fi, cellular, satellite, and other well-known technologies. The first user device 14a is configured to display an offline notification 84 (see FIG. 4) when the first user device 14a is offline (e.g. not connect to the network 22). In certain aspects, the at least one user device 14 is configured to display a QR code such that a mobile device associated with the user profile 64 associated with the at least one user device 14 can scan the QR code to authenticate the mobile device for usage in connection with the user profile 64.

The at least one personal care partner device 16 is configured to communicate with the first user device 14a via the care management server 10. The at least one personal care partner device 16 is configured to display selected information 86 (see FIG. 4F) of the user profile 64 for the personal care partner who is assigned to the at least one personal care partner device 16 can conveniently access and view the selected information 86 to efficiently provide personal support and guidance to the user of the first user device 14a. The at least one personal care partner device 16 is configured to create events 88 (see FIG. 4F) for display on the calendar 78 on the first user device 14a. The at least one personal care partner device 16 is configured to monitor and update the events 88. The at least one personal care partner device 16 is configured to communicate with the first user device 14a via videotelephony and is configured to initiate communication between the first user device 14a and other devices on the network 22, such as the at least one provider device 18, the at least one companion device 20, and the emergency medical server 12, via videotelephony (e.g., teleconferencing) while also being on the communication (e.g, video call) and can optionally drop off the video call such that the first user device 14a is still in communication with the other device.

The at least one personal care partner device 16 is configured to display situational call scripts 90 (see FIG. 4F) to prompt the personal care partner with dialogue to discuss with the user of the first user device 14a. The at least one personal care partner device 16 is configured to receive call notes 92 (see FIG. 4F) entered by the personal care partner to document items of importance discussed during the video call with the user of the first user device 14a.

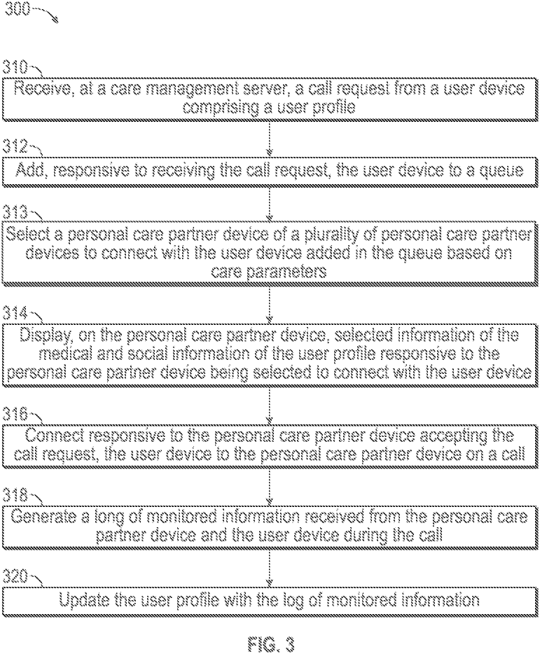

FIG. 3 illustrates an example process 300 using the care management server 10, the first user device 14a, and the at least one personal care partner device 16 of FIG. 2. While FIG. 3 is described with reference to FIG. 2, it should be understood that the process steps of FIG. 3 may be performed by other systems.

The process begins by proceeding to step 310 when the processor 36 of the care management server 10 a call request from the first user device 14a comprising the user profile 64. The user profile comprises the medical information 66 and the social information 68 of the user associated with the first user device 14a. As depicted at step 312, the processor 36 of the care management server 10 adds, responsive to receiving the call request, the first user device 14a to the queue 67. At step 313, the processor 36 of the care management server 10 selects a personal care partner device of a plurality of personal care partner devices (e.g., the at least one personal care partner device 16) to connect with the first user device 14a added to the queue 67 based on care parameters. At step 314, the processor 36 of the care management server 10 displays, on the personal care partner device (e.g., the at least one personal care partner device 16), selected information of the medical information 66 and the social information 68 of the user profile 64 responsive to the personal care partner device (e.g., the at least one personal care partner device 16) being selected to connect with the first user device 14a. The processor 36 of the care management server 10 connects, responsive to the at least one personal care partner device 16 accepting the call request, the first user device 14a to the at least one personal care partner device 16 on a call, as illustrated at step 316.

As depicted at step 318, the processor 36 of the care management server 10 generates the log 69 of monitored information 94 received from the at least one personal care partner device 16 and the first user device 14a during the call. As depicted at step 320, the processor 36 of the care management server 10 updates the user profile 64 with the log 69 of monitored information 94.

FIG. 3 sets forth the example process 300 using the care management server 10, the first user device 14a, and the at least one personal care partner device 16 of FIG. 2. An example will now be described with reference to the example process of FIG. 3 and the example illustrations in FIG. 4A-4EF

With particular reference to FIG. 4A, the first user device 14a (e.g., the at least one user device 14) is located at a user environment, such as a home or room at a living community, for example, of the user associated with the user profile 64 that is uniquely associated to the first user device 14a. The first user device 14a is stationary with respect to the user environment such that the user can securely use the first user device 14 independently of entering a password due to the user profile 64 being uniquely associated to the first user device 14. In such a manner, the first user device 14a acts as a passwordless device. In certain aspects, the care management server 10 is configured to receive the location of the first user device 14a, via the location device 51, to confirm that the first user device 14a is at the user environment associated with the user profile 64 associated with the first user device 14a.

The user associated with the first user device 14a can initiate a call request to interact with a personal care partner by engaging the call personal care partner button 80. The user may wish to interact with the personal care partner for a number of reasons including, but not limited to, scheduling appointments, scheduling telemedicine visits, scheduling in-home doctor visits, scheduling in office doctor visits with a primary care doctor or a specialist, scheduling transportation, scheduling meal deliveries, scheduling grocery delivery, scheduling medication intake, placing or refilling medications at a pharmacy, talking about mental/emotional concerns, talking about management of everyday life, talking about health concerns, asking questions, requesting to speak with a provider, and discussing other health or social related concerns, to name a few examples.

Referring to FIG. 4E, the care management server 10 receives the call request, responsive to the call personal partner button 80 being engaged, and adds the call request to the queue 67. As shown in FIG. 4B, while the first user device 14a is waiting in the queue 67, a connecting display 96 is displayed on the first user device 14a to notifying the user that is being connecting to a personal care partner. In certain aspects, the connecting display 96 also displays an estimated wait time. With the first user device 14a added to the queue 67 and waiting to connect with a personal care partner, the care management server 10 evaluates the user profile 64 associated with the first user device 14a for selecting or determining which personal care partner associated with one of the personal care partner devices of the at least one personal care partner device 16 is appropriate for handling the user associated with the first user device 14a. The care management server 10 selects or determines the appropriate personal care partner based on the user profile 64 as well as on a variety of care parameters including, but not limited to, skills of the personal care partner associated with the least one personal care partner device 16 with respect to the user profile 64 of the user of the first user device 14a, needs of the user associated with the first user device 14a identified in the user profile 64, urgency of the call request, and other parameters that facilitate routing of the first user device 14a to the appropriate personal care partner via the at least one personal care partner device 16.

With the appropriate personal care partner selected, and prior to connecting the at least one personal care partner device 16 associated with the appropriate personal care partner that is selected, the care management server 10 displays on the at least one personal care partner device 16 selected information of the medical information 66 and the social information 68 of the user profile 64, as depicted in FIG. 4F. Displaying the selected information of the medical information 66 and the social information 68 of the at least one personal care partner device 16 allows the personal care partner to review and become acquainted or re-acquainted with the user associated with the information to facilitate personalized support and guidance.

Responsive to the personal care partner accepting the call request via the at least one personal care partner device 16, the care management server 10 connects the first user device 14a to the at least one personal care partner device 16, as depicted in FIG. 4B. For privacy between the first user device 14a and the at least one personal care partner device 16 during the call, the camera of the first user device 14a can be selectively turned off by either the first user device 14a or the at least one personal care partner device 16a. Volume on the first user device 14a can be selectively controlled via user device control volume buttons 102. Similarly, volume on the at least one personal care partner device 16a can be selectively controlled via partner device volume button 104. The at least one personal care partner device 16a can also mute the call via a mute button 106 and can control (e.g., enable and disable) the video via a video button 108.

During the call, the care management server 10 generates the log 69 received from monitored information 94 input into the at least one personal care partner device 16 including the call notes 92. The care management server 10 updates the user profile 64 with the log 69 of the monitored information 94.

While on the call, the at least one care partner device 16a can initiate holding the call via a hold button 110, initiate a provider device of the at least one provider device 18 or a companion device of the at least on companion device 20 to join the call via a add caller button 112, and initiate transferring the first user device 14a to one of the at least one provider device 18 and the at least one companion device 20 via a transfer button 114. The at least one care partner device 16a can also initiate connecting or transferring the first user device 14a to the emergency medical service server 12 via a call 911 button 116.

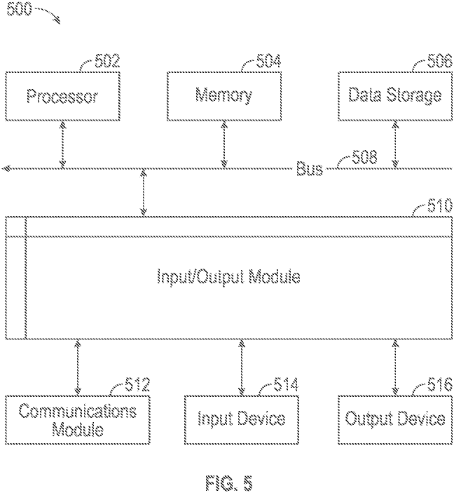

FIG. 5 is a block diagram illustrating an example computer system 500 with which the care management server 10, the emergency medical service server 12, the electronic medical record server 13, the at least one user device 14, such as the first user device 14a, the at least one personal care partner device 16, the at least one provider device 18, and the at least one companion device 20 of FIG. 2 can be implemented. In certain aspects, the computer system 500 may be implemented using hardware or a combination of software and hardware, either in a dedicated server, or integrated into another entity, or distributed across multiple entities.

Computer system 500 (e.g., the care management server 10, the emergency medical service server 12, the electronic medical record server 13, the at least one user device 14, such as the first user device 14a, the at least one personal care partner device 16, the at least one provider device 18, and the at least one companion device 20) includes a bus 508 or other communication mechanism for communicating information, and a processor 502 (e.g., the processor 36, 40, 41, 44, 52, 56, 60) coupled with bus 508 for processing information. According to one aspect, the computer system 500 can be a cloud computing server of an IaaS that is able to support PaaS and SaaS services.

Computer system 500 can include, in addition to hardware, code that creates an execution environment for the computer program in question, e.g., code that constitutes processor firmware, a protocol stack, a database management system, an operating system, or a combination of one or more of them stored in an included memory 504 (e.g., the memory 38, 42, 43, 46, 54, 58, 62), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 508 for storing information and instructions to be executed by processor 502. The processor 502 and the memory 504 can be supplemented by, or incorporated in, special purpose logic circuitry.

The instructions may be stored in the memory 504 and implemented in one or more computer program products, e.g., one or more modules of computer program instructions encoded on a computer readable medium for execution by, or to control the operation of, the computer system 500.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 500 further includes a data storage device 506 such as a magnetic disk or optical disk, coupled to bus 508 for storing information and instructions. Computer system 500 may be coupled via input/output module 510 to various devices. The input/output module 510 can be any input/output module. Example input/output modules 510 include data ports such as USB ports. In addition, input/output module 510 may be provided in communication with processor 502, so as to enable near area communication of computer system 500 with other devices. The input/output module 510 may provide, for example, for wired communication in some implementations, or for wireless communication in other implementations, and multiple interfaces may also be used. The input/output module 510 is configured to connect to a communications module 512. Example communications modules 512 (e.g., the communications module 24, 26, 27, 28, 20, 32, 34) include networking interface cards, such as Ethernet cards and modems.

In certain aspects, the input/output module 510 is configured to connect to a plurality of devices, such as an input device 514 (e.g., the input device 48) and/or an output device 516 (e.g., the output device 50). Example input devices 514 include a keyboard, a touch screen, and a pointing device, e.g., a mouse or a trackball, by which a user can provide input to the computer system 500. Other kinds of input devices 514 can be used to provide for interaction with a user as well, such as a tactile input device, visual input device, audio input device, or brain-computer interface device.

According to one aspect of the present disclosure the care management server 10, the emergency medical service server 12, the electronic medical record server 13, the at least one user device 14, such as the first user device 14a, the at least one personal care partner device 16, the at least one provider device 18, and the at least one companion device 20, can be implemented using a computer system 500 in response to processor 502 executing one or more sequences of one or more instructions contained in memory 504. Such instructions may be read into memory 504 from another machine-readable medium, such as data storage device 506. Execution of the sequences of instructions contained in main memory 504 causes processor 502 to perform the process steps described herein. One or more processors in a multi-processing arrangement may also be employed to execute the sequences of instructions contained in memory 504. Processor 502 may process the executable instructions and/or data structures by remotely accessing the computer program product, for example by downloading the executable instructions and/or data structures from a remote server through communications module 512 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

The term “machine-readable storage medium” or “computer-readable medium” as used herein refers to any medium or media that participates in providing instructions or data to processor 502 for execution. The term “storage medium” as used herein refers to any non-transitory media that store data and/or instructions that cause a machine to operate in a specific fashion. Such a medium may take many forms, including, but not limited to, nonvolatile media, volatile media, and transmission media.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 508. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

In one aspect, a method may be an operation, an instruction, or a function and vice versa. In one aspect, a clause or a claim may be amended to include some or all of the words (e.g., instructions, operations, functions, or components) recited in either one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. §112, sixth paragraph, unless the element is expressly recited using the phrase “means for” or, in the case of a method claim, the element is recited using the phrase “step for”.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A computer-implemented method for providing personalized patient support and guidance based on secured medical and social information, the method comprising:
receiving, at a care management server, a call request from a user device comprising a user profile, wherein the user profile comprises medical and social information of a user associated with the user device;
adding, responsive to receiving the call request, the user device to a queue;
selecting a personal care partner device of a plurality of personal care partner devices to connect with the user device added in the queue based on care parameters;
displaying, on the personal care partner device, selected information of the medical and social information of the user profile responsive to the personal care partner device being selected to connect with the user device;
connecting, responsive to the personal care partner device accepting the call request, the user device to the personal care partner device on a call;
generating a log of monitored information received from the personal care partner device and the user device during the call; and
updating the user profile with the log of monitored information.

2. The computer-implemented method of claim 1, further comprising:
receiving, from the personal care partner device, instructions to connect the user device in communication with a provider device; and
connecting, responsive to receiving the instructions to connect the user device in communication with the provider device, the provider device to join the call.

3. The computer-implemented method of claim 2, further comprising:
disabling the personal care partner device from the call while maintaining communication between the user device and the provider device.

4. The computer-implemented method of claim 1, further comprising:
receiving, from the personal care partner device, instructions to connect the user device in communication with an emergency medical service server; and
connecting, responsive to receiving the instructions to connect the user device in communication with the provider device, the emergency medical service server to join the call.

5. The computer-implemented method claim 1, further comprising:
accessing prior to receiving the call request, from an electronic medical record server, an electronic medical record associated with the user of the user device; and
integrating information of the electronic medical record associated with the user into the user profile.

6. The computer-implemented method of claim 1, further comprising:
performing analytics on information associated with the user profile; and
updating the user profile with results of the analytics.

7. The computer-implemented method of claim 1, further comprising:
determining a persona of the user of the user device based on the medical information and the social information; and
associating the persona of the user with the user profile associated with the user device.

8. The computer-implemented method of claim 1, further comprising:
associating the user profile exclusively to the user device to securely enable identification, absent password entry, of the user device when receiving the call request.

9. A system comprising:
a memory comprising instructions; and
a processor configured to execute the instructions which, when executed, cause the processor to:
receive, at a care management server, a call request from a user device comprising a user profile, wherein the user profile comprises medical and social information of a user associated with the user device;
add, responsive to receiving the call request, the user device to a queue;
select a personal care partner device of a plurality of personal care partner devices to connect with the user device added in the queue based on care parameters;
display, on the personal care partner device, selected information of the medical and social information of the user profile responsive to the personal care partner device being selected to connect with the user device;
connect, responsive to the personal care partner device accepting the call request, the user device to the personal care partner device on a call;
generate a log of monitored information received from the personal care partner device and the user device during the call; and
update the user profile with the log of monitored information.

10. The system of claim 9, wherein the instructions further cause the processor to:
receive, from the personal care partner device, instructions to connect the user device in communication with a provider device; and
connect, responsive to receiving the instructions to connect the user device in communication with the provider device, the provider device to join the call.

11. The system of claim 10, wherein the instructions further cause the processor to:
disable the personal care partner device from the call while maintaining communication between the user device and the provider device.

12. The system of claim 9, wherein the instructions further cause the processor to:
receive, from the personal care partner device, instructions to connect the user device in communication with an emergency medical service server; and
connect, responsive to receiving the instructions to connect the user device in communication with the provider device, the emergency medical service server to join the call.

13. The system of claim 9, wherein the instructions further cause the processor to:
access prior to receiving the call request, from an electronic medical record server, an electronic medical record associated with the user of the user device; and
integrate information of the electronic medical record associated with the user into the user profile.

14. The system of claim 9, wherein the instructions further cause the processor to:
perform analytics on information associated with the user profile; and
update the user profile with results of the analytics.

15. The system of claim 9, wherein the instructions further cause the processor to:
determine a persona of the user of the user device based on the medical information and the social information; and
associate the persona of the user with the user profile associated with the user device.

16. The system of claim 9, wherein the instructions further cause the processor to:
associate the user profile exclusively to the user device to securely enable identification, absent password entry, of the user device when receiving the call request.

17. A non-transitory machine-readable storage medium comprising machine-readable instructions for causing a processor to execute a method, the method comprising:
receiving, at a care management server, a call request from a user device comprising a user profile, wherein the user profile comprises medical and social information of a user associated with the user device;
adding, responsive to receiving the call request, the user device to a queue;
selecting a personal care partner device of a plurality of care partner devices to connect with the user device added in the queue based on care parameters;
displaying, on the personal care partner device, selected information of the medical and social information of the user profile responsive to the personal care partner device being selected to connect with the user device;
connecting, responsive to the personal care partner device accepting the call request, the user device to the personal care partner device on a call;
generating a log of monitored information received from the personal care partner device and the user device during the call;
updating the user profile with the log of monitored information;
accessing, from an electronic medical record server, an electronic medical record associated with the user of the user device; and
integrating information of the electronic medical record associated with the user into the user profile.

18. The non-transitory machine-readable storage medium of claim 17, further including instructions for causing the processor to execute the method comprising:
receiving, from the personal care partner device, instructions to connect the user device in communication with a provider device; and
connecting, responsive to receiving the instructions to connect the user device in communication with the provider device, the provider device to join the call.

19. The non-transitory machine-readable storage medium of claim 17, further including instructions for causing the processor to execute the method comprising:
performing analytics on information associated with the user profile; and
updating the user profile with results of the analytics.

20. The non-transitory machine-readable storage medium of claim 17, further including instructions for causing the processor to execute the method comprising:
associating the user profile exclusively to the user device to securely enable identification, absent password entry, of the user device when receiving the call request.
